(19) United States
(12) Patent Application Publication
(10) Pub. No.: US 2026/0218153 A1
(43) Pub. Date: Jul. 30, 2026

(54) NOVEL ENDO-B-N-ACETYLGLUCOSAMINIDASES AND THEIR USES FOR THE PRODUCTION OF FREED N-GLYCANS FROM FOOD

(71) Applicant: Board of Regents of the Nevada System of Higher Education on behalf of the University of Nevada, Ren, Reno, NV (US)

(72) Inventors: Steve Frese, Reno, NV (US); Sercan Karav, Aksehir (TR); Matthew Bolino, Reno, NV (US)

(21) Appl. No.: 19/464,955

(22) Filed: Jan. 30, 2026

Related U.S. Application Data
(60) Provisional application No. 63/751,370, filed on Jan. 30, 2025.

(51) Int. Cl. C12N 9/24; A61K 38/00; A61P 1/14

(52) U.S. Cl. CPC C12N 9/2402; A61P 1/14; C12Y 302/01096; A61K 38/00

(57) ABSTRACT

Disclosed herein are novel recombinant endo-β-N-acetylglucosaminidases enzymes and methods of using endo-β-N-acetylglucosaminidases enzymes.

### CROSS-REFERENCE TO RELATED APPLICATIONS

This application claims the benefit under 35 USC § 119(e) of U.S. Provisional Patent Application No. 63/751,370, filed Jan. 30, 2025, which is herein incorporated by reference.

### FEDERALLY SPONSORED RESEARCH OR DEVELOPMENT

This invention was made with Government support under Agreement No. GRANT-13385133, awarded by the United States Department of Agriculture, National Institute of Food and Agriculture. The United States Government has certain rights in the invention.

### REFERENCE TO A SEQUENCE LISTING

The Sequence Listing XML file filed herewith, titled UNR-24-020_Sequence, created on Jan. 28, 2026, and containing 16,299 bytes, is herein incorporated by reference.

### FIELD

The present disclosure relates to novel recombinant enzymes.

### BACKGROUND

The gut microbiome is a dynamic and dense ecosystem containing a diverse and dynamic assembly of microbes. This community encounters a wide array of distinct carbohydrate substrates that represent the most abundant, common, and highest energy resource for the distal gut microbiome. As most host animals are able to degrade and absorb simple carbohydrates such as sucrose or lactose, only the relatively complex carbohydrate structures escape digestion and absorption by the host and transit the gut to the distal large intestine. Once in the large intestine, these substrates are a major source of energy for bacterial fermentation, resulting in the production of metabolites such as short-chain fatty acids.

Prior to fiber fermentation by gut microbes, a wide variety of microbiota-harboring enzymes known as glycoside hydrolases (GHs), a subset of enzymes within the group of carbohydrate-active enzymes (CAZymes), act on glycosidic bonds to depolymerize fibers into smaller subunits for further metabolism. Most gut microbes, such as the bacteria found within the Bacteroidetes phylum, organize there CAZymes into gene clusters known as polysaccharide utilization loci (PULs), which typically target a specific dietary glycan. Currently, there are over 180 GH families targeting distinct glycosidic bonds through various mechanisms and different specificities.

When dietary fiber intake is low or absent, microbes prioritize different substrates present in the colonic environment, such as undigested dietary fats or proteins, as well as endogenous host-derived proteins and glycoproteins, such as mucins. Importantly, fiber consumption is strongly linked to improvements in health outcomes, likely in part because of the reorganization of nutritional priorities of gut microbes. Several large, well powered population-level studies have identified associative links to dietary fiber intake and morbidity and, more recently, mechanistic studies have begun to link the consumption of fiber with microbiome metabolism of these undigested glycans.

However, current interest in fiber supplements and prebiotics has focused primarily on polymers of varying chain lengths based on inulin (fructooligosaccharides, FOS), galactooligosaccharides (GOS), polymers of dextrose (polydextrose, PDX), or resistant starches (RS). In contrast, N-glycans are complex oligosaccharides composed of a wider variety of monomers, with varying degrees of polymerization and terminal monosaccharides that are of limited accessibility by many gut bacteria. They are abundantly found conjugated to dietary proteins like soy, milk, pea, egg white, wheat, and others. The relative structural and compositional complexity offers an improved palette of selective, bioactive glycans to query specificity in enriching for beneficial bacteria in the gut microbiome, such as Bifidobacterium, Lactobacillus, or Akkermansia.

Much of the focus in the field has centered on the use of structurally homogenous fibers as supplements, rather than increasing the structural diversity of effective bioactive fibers already found in foods, but which are poorly utilized by many bacteria because of their conjugation to proteins. N-glycans, when separated from a polypeptide, act as effective prebiotics with better activity than an intact N-glycoprotein, and orthologous evidence has demonstrated that access to N-glycans is an important ecological strategy for beneficial microbes in the human gut microbiome. Enzymes that more effectively release N-glycans from foodstuffs are needed.

### BRIEF SUMMARY

Disclosed herein are novel recombinant endo-β-N-acetylglucosaminidases (ENGases) enzymes and methods of use thereof. Further disclosed herein are compositions comprising novel recombinant ENGases enzymes.

In one aspect, the present disclosure provides for a recombinant protein. In some embodiments, the recombinant protein comprises the amino acid sequence of SEQ ID NO: 4. In some embodiments, the recombinant protein comprises the amino acid sequence of SEQ ID NO: 5. recombinant protein comprises the amino acid sequence of SEQ ID NO: 6.

In some embodiments, the recombinant protein is an enzyme capable of releasing or cleaving N-glycans from glycoproteins.

In one aspect, the present disclosure provides for methods of using the presently disclosed recombinant ENGase enzymes.

In some embodiments, the method comprises orally ingesting an enzyme and one or more foodstuffs to increase the released N-glycan content of the one or more foodstuffs during digestion, wherein the enzyme comprises the amino acid sequence of SEQ ID NO: 4, and wherein the one or more foodstuffs comprise one or more N-glycoproteins.

In some embodiments, the method comprises orally ingesting an enzyme and one or more foodstuffs to increase the released N-glycan content of the one or more foodstuffs during digestion, wherein the enzyme comprises the amino acid sequence of SEQ ID NO: 5, and wherein the one or more foodstuffs comprise one or more N-glycoproteins.

In some embodiments, the method comprises orally ingesting an enzyme and one or more foodstuffs to increase the released N-glycan content of the one or more foodstuffs during digestion, wherein the enzyme comprises the amino acid sequence of SEQ ID NO: 6, and wherein the one or more foodstuffs comprise one or more N-glycoproteins.

In one aspect, the present disclosure provides for compositions comprising the presently disclosed recombinant enzymes and a second compound, for example, a glycoprotein, or a food containing a glycoprotein.

In some embodiments, the composition comprises the recombinant enzyme according to the amino acid sequence of SEQ ID NO: 3, 4, or 5, and a glycoprotein. In some embodiments, the composition comprises the recombinant enzyme according to the amino acid sequence of SEQ ID NO: 3, 4, or 5, and a food containing a glycoprotein. In some embodiments, the glycoprotein comprises high mannose.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges are encompassed within the scope of the present disclosure.

### BRIEF DESCRIPTION OF THE DRAWINGS

FIG. 1 shows protein sequence alignments identifying 14 discrete ENGases with each group exhibiting identical sequence alignments.

FIG. 2 shows ENGase activity determined by RNAseB gel-shift assay; from left to right is the protein ladder, negative control of intact RNAseB, EndoBI-1-treated RNAseB, RNAseB incubated with AVUL01; RNAseB incubated with BCA01, and RNAseB incubated with BFIN01.

FIG. 3A shows that the presently disclosed enzymes each release N-glycans from lactoferrin (LF) and horseradish peroxidase (HRP) with significantly more N-glycans released after 45 mins of incubation under each enzyme's ideal conditions when LF is the substrate, except in the case of BCA01, which is not inhibited by a 1,3 core fucosylation of N-glycans.

FIG. 3B shows that while all three enzymes release comparable amounts of N-glycans from LF (P>0.05), only BCAC01 shows a trend toward elevated N-glycan release on HRP (P=0.051).

### DETAILED DESCRIPTION

It is to be understood that this disclosure is not limited to particular methods and experimental conditions described, as such methods and conditions may vary. It is also to be understood that the terminology used herein is for describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

### General Overview

The present disclosure provides novel recombinant endo-β-N-acetylglucosaminidases (ENGases) enzymes that release N-glycans from dietary N-glycoproteins. These recombinant enzymes provide optimum utility under different food processing conditions (e.g., temperature and/or pH).

### Novel ENGase Enzymes

Embodiments of the present disclosure include the ENGases enzymes translated from presently disclosed nucleic acid sequences of SEQ ID NOs: 1-3, and ENGases enzymes corresponding to the amino acid sequences of SEQ ID NOs: 4-6. The nucleic acid sequence of the recombinant ENGase enzymes disclosed herein, e.g., SEQ ID NOs:1-3, can each be molecularly tagged at the terminus of the sequence for purification processes. Examples of molecular tags include SEQ ID NOS: 6-9, i.e., His tags, GST tags, or SUMO tags.

### Methods of Using ENGase Enzymes

Embodiments of the present disclosure include the use of recombinant ENGases in methods of processing foodstuffs and improving the nutritional profile of food products.

Specifically, these enzymes can be used to increase the release of N-glycans from glycopeptides.

### Compositions Comprising Novel ENGase Enzymes

The presently disclosed recombinant ENGases can be used in compositions comprising glycopeptides or in combination with glycopeptides. In some examples, the glycopeptides comprise high mannose and/or hybrid N-glycan.

### EXAMPLES

3.1 Sample origins. This study was conducted in Nevada (USA) and was overseen by the University of Nevada, Reno Institutional Review Board (IRB) under approval #1751022. Adult participants were recruited from email listservs, local flyers at businesses, radio and television interviews, and other print and online media solicitations. All methods were carried out in accordance with the relevant guidelines and regulations outlined by the Declaration of Helsinki. Adult residents of Nevada interested in participating in the study were pre-screened using an online intake portal to self-affirm that they met all of the inclusion criteria and none of the exclusion criteria prior to being consented and enrolled into the study. Informed consent was obtained from adults (over 18 years of age) who self-identified that they met the study inclusion criteria. Study inclusion criteria included: (1) over 18 years of age, (2) no known and untreated infection (e.g., SARS-CoV-2), (3) individuals were consuming an ad libitum diet without medical intervention and (4) individuals were literate in English and/or Spanish. Individuals were excluded from the study if they: (1) had an active untreated infection within the last two weeks (e.g., SARS-CoV-2); (2) had a recent diagnosis of a malarial or parasitic infection; (3) were unable to stool spontaneously or had medically altered GI function (e.g., an ileostomy or colostomy); (4) were receiving a medically prescribed diet; (5) were currently hospitalized, critically ill, or were unable to give informed consent. Participants agreed to complete surveys of dietary intake and lifestyle habits, as well as demographic information. Additionally, participants agreed to provide a fecal sample.

Participants were instructed to return fecal samples so they could be processed and stored at −80 C within 4 hours of defecation. ~100 mg of fecal sample was collected in ZymoResearch DNA/RNA Shield and stored as directed for subsequent DNA extraction at −80 C. DNA was then extracted using a ZymoBIOMICS DNA MiniPrep kit (ZymoResearch, Irvine, CA USA) according to the manufacturer's instructions, which included a homogenization step using a FastPrep 24 homogenizer (MP Biomedicals).

3.2. Sequencing and identification of endo-α-N-acetylglucosaminidases (ENGases). A total of 18 samples were subjected to whole genomic DNA was prepared by the University of Nevada, Reno Genome Center using an Illumina DNA library preparation kit for sequencing on a NextSeq 2000 with 150 bp paired-end reads. Sequencing data was demultiplexed and reads were quality controlled and adapters were removed using bbduk. Reads from each sample were assembled using metaSPADES and the resulting assemblies were binned using metaBAT2. Binned assemblies were then annotated using Prokka and CAZYmes were identified using dbcan2 to identify candidate ENGases as members of the GH18 CAZyme family (elsewhere annotated as RENDA:EC3.2.1.96). All predicted ENGases were then aligned using CLUSTALW and identical sequences were identified and de-replicated for further analysis. Taxonomic identification of the de-replicated protein sequences was then performed using blastx against the NCBI database ( ) and comparing the highest ranked matches.

3.3 Cloning and production of candidate enzymes. De-replicated candidate sequences were then selected for cloning and expression. Primers were designed to amplify the predicted sequences, omitting transmembrane domains, signal peptides, or predicted anchor domains, if present. These primers included a complementary overhang enabling in-frame cloning into p15TV-L. Expression from this system results in an in-frame N-terminal histidine tag and a TEV-cleavage site to remove the histidine tag, under the regulation of a T7 promoter.

Candidates ENGases were then amplified using the conditions and primers outlined in Table 1. For all reactions, amplification was performed using 2 μL of DNA obtained from the original fecal sample subjected to sequencing diluted 1:10 in 10 mM Tris Cl, pH 8.0. The reactions were carried out using Phusion High-Fidelity PCR Master Mix with HF buffer (New England Biolabs, Ipswich, MA USA) and 1 μL of 10 μM of each primer for the respective reactions. Amplification of desired PCR products were confirmed in an agarose gel, and the resulting amplicon was purified using a ZymoResearch Clean and Concentrator-25 kit (ZymoResearch, Irvine, CA USA).

BseRI-digested p15TV-L and the purified gene of interest were assembled using NEBuilder HiFi DNA Assembly Master Mix (New England Biolabs, Ipswich, MA USA) and then transformed into T7 Express High Efficiency E. coli (New England Biolabs, Ipswich, MA USA) following the manufacturer's instructions and then serially diluted for plating on LB agar containing carbenicillin (100 μg/mL). After incubating at 37 C overnight, colonies were selected at random and subcultured, then grown in LB media containing carbenicillin overnight at 37 C, with shaking at 250 RPM. An aliquot of cells were subjected to plasmid extraction with a ZR Plasmid Miniprep Classic kit (ZymoResearch, Irvine, CA USA). Plasmid identity was confirmed by restriction digestion against the predicted plasmid sequence. One clone with the correct plasmid sequence for each protein of interest was stored at −80 C in 15% glycerol, and remaining intact plasmid DNA was stored at −20° C.

Cell lines were cultured on LB containing carbenicillin (100 μg/mL) and grown aerobically overnight at 37 C and 250 RPM before adding 3 volumes of Terrific Broth with added lactose (7.6 g/L). After expansion overnight at 37 C, cells were harvested by centrifugation, sonicated, and purified using a Ni-resin column. Raw cell lysate and the purified protein were examined using SDS-PAGE, against the predicted size of the protein of interest. Confirmed protein then underwent buffer exchange to remove residual imidazole and stored at standardized concentrations in 50% glycerol.

3.4 Characterization of enzyme activity. The enzymatic activity of AVUL01, BCAC01, and BFIN01 was characterized as previously described in Karav, Le Parc, et al., 2015. Briefly, a total reaction volume (30 μL) consisting of 10 μL of enzyme (500 ng/μL in 50% glycerol), 3 μL of 200 mM sodium phosphate buffer (adjusted to pH 5, 6, or 7), 2 μL of deionized water, and 15 μL of bovine lactoferrin (10 mg/mL). Reactions were performed in triplicate for each enzyme across reported temperatures (24, 30, 37, or 43° C.) and pH levels (pH 4, 5, 6, or 7). Controls included substrate without enzyme. The reaction mixtures were incubated for 16 hours under the specified conditions and the reaction ended and the release of N-glycans was quantified by precipitating protein with 2 volumes of ice-cold ethanol. The plates were then incubated at −80° C. for one hour or more, and then centrifuging the samples, recovering the supernatant, and quantifying the residual glycans after drying to remove the ethanol, using a plate-based phenol sulfuric assay.

3.5 Statistical analyses. Response Surface Methodology (RSM) was utilized to evaluate the interactive effects of temperature and pH on glycan release and determine the optimal conditions for each enzyme, using temperature and pH as factors. The rsm package was used to conduct this analysis. Statistical tests were conducted as noted, using the rstatix package with a false discovery rate (FDR) correction for multiple comparisons, where indicated.

### Results

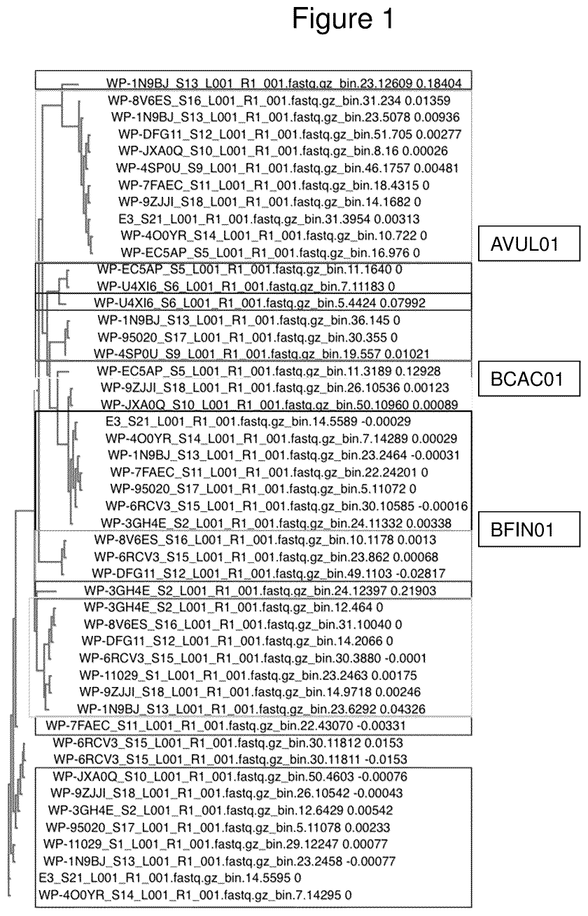

4.1 Identification and production of novel endo-β-N-acetyl-glucosaminidases from human fecal samples. After assembly and annotation, sequences identified as candidate ENGases were aligned and fourteen distinct sequences were identified from the dataset. After collapsing identical sequences, the centermost representative sequence chosen for characterization (FIG. 1). Then, having analyzed the sequences, we identified three sequences with likely ENGase activity and selected these for cloning and expression, after removing protein domains expected to limit protein expression from the sequences assembled from the original sequences generated from the fecal samples. The resulting sequences, SEQ ID NOs: 1-3, represent these novel sequences used for cloning and expression.

<<SEQ ID NO: 1>> When comparing the translated amino acid sequence, SEQ ID NO: 1 (AVUL01) was predicted as originating from Alistipes onderdonkii subsp. vulgaris, a member of the Rikenellaceae family, and the nucleotide sequence was reported in the first characterization of another strain of this species in 2020. No other significant nucleotide sequence matches are found in the NCBI database. These nucleotide sequence matches bear high sequence homology, but these sequences have not been characterized to determine their specific functions and characteristics.

The closest amino acid sequence match to AVUL01 that has been functionally characterized originates from Bacteroides thetaiotaomicron but that protein is less than 63% similar, suggesting that while these two protein sequences may have some functional homology, there is no evidence that they have matching enzymatic characteristics. Further, the enzymes described here (SEQ ID NOs: 4-6) have been modified from their original natural sequence to remove protein domains anticipated to be unnecessary for function and to improve production of the enzymes in vitro.

<<SEQ ID NO: 2>> SEQ ID NO: 2 (BCAC01) is predicted to originate from Bacteroides ovatus or B. xylanisolvens, both members of the Bacteroidaceae family. These nucleotide sequence matches bear high sequence homology, but these sequences are only predictive and the resulting enzymes have not been characterized to determine their specific functions and characteristics. Similarly, the closest predicted amino acid sequences are also found among B. ovatus and B. xylanisolvens, but these proteins have not been characterized.

To produce SEQ ID NO: 2 as a protein from the predicted sequences, the nucleotide sequence was modified from its original sequence to remove protein domains we anticipated to be unnecessary for function and to improve production of the enzymes in vitro.

<<SEQ ID NO: 3>> SEQ ID NO: 3 (BFIN01) was found to have significant sequence homology with a nucleotide sequence in the Bacteroides finegoldii genome only, with no other significant sequence matches. When comparing the predicted amino acid sequence to the NCBI database, there were only significant matches within the B. finegoldii and B. stercoris genomes, with all other results bearing less than 75% sequence homology, indicating that the characteristics of this predicted protein sequence is likely to be significantly different from even other homologs across Bacteroides.

To produce SEQ ID NO: 3 as a protein from the predicted sequences, the nucleotide sequence was modified from its original sequence to remove protein domains we anticipated to be unnecessary for function and to improve production of the enzymes in vitro.

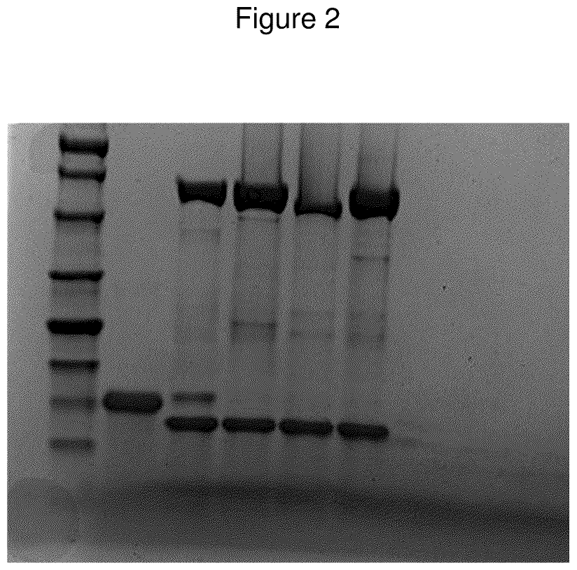

4.2 Endo-α-N-acetylglucosaminidases cleave N-_glycan from RNase B. After cloning and expression in E. coli, followed by purification using a Ni column (see Methods), we compared the ability of these novel proteins (AVUL01, BCAC01, and BFIN01) to remove a single high mannose N-glycan from RNAse B, demonstrating ENGase activity. When we tested these proteins against a previously described ENGase (EndoBI-1), under previously published conditions, we find that these three novel proteins demonstrate effective ENGase activity and their apparent activity is greater than EndoBI-1 (FIG. 2).

4.3. Optimization on the release of N-glycans from bovine lactoferrin and horseradish peroxidase from endo-α-N-acetyl_glucosaminidases. To determine the optimum conditions for the activity of these enzymes, we tested the release of N-glycans from bovine lactoferrin (bLF) under different temperatures (24, 30, 37, and 43 C) and at different pH values (pH 4, 5, 6, and 7). Using response surface methodology in the r package rsm, we determined the optimum conditions for each of the enzymes given a complex N-glycoprotein substrate. The optimum conditions, determined using response surface methodology, are reported in TABLE 1, in comparison to EndoBI-1, a previously described ENGase and PNGaseF, a commercial ENGase used in molecular biology applications.

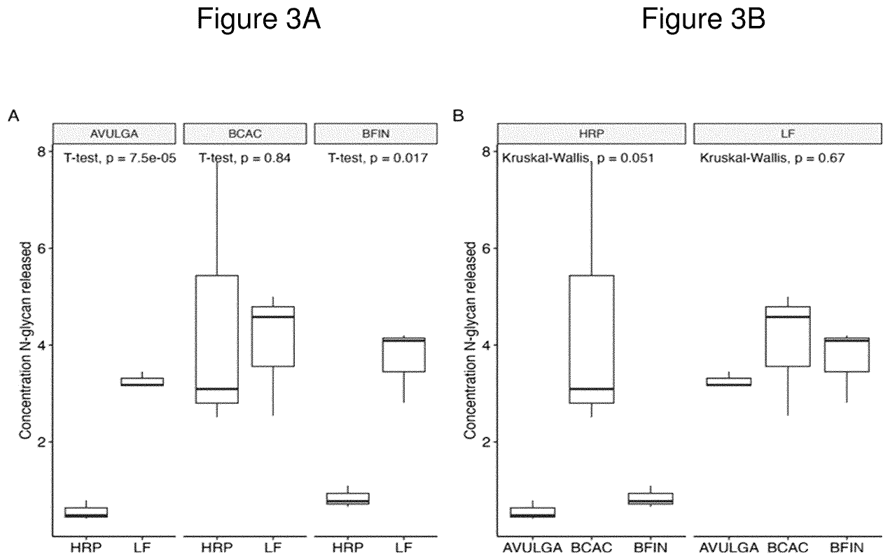

Using these optimized conditions, we tested the activity of these enzymes on both a complex, animal-derived N-glycoprotein (bLF) and a plant-derived, α-1,3 core-fucosylated high mannose N-glycoprotein, horseradish peroxidase (HRP). Under the same conditions, we found that while BCAC01 and BFIN01 could cleave the N-glycans in bLF, only BCAC01 could cleave the N-glycans from HRP, demonstrating strong differences in the total N-glycan released under optimized conditions (TABLE 1). In the case of AVUL01 and BFIN01, the release of N-glycans on bLF was significantly greater than on HRP, while BCAC01 showed no reduction in N-glycan release (P>0.05, t-test). This finding confirmed that while the predicted GH18 family activity of these enzymes is indeed present and responsible for the release of N-glycans from N-glycoproteins, their specificities are differentially affected by α-1,3 core fucosylation of N-glycans. In contrast, BCAC01 showed no loss of activity on α-1,3 core fucosylated N-glycans. The amount of released glycan was comparable across all three enzymes, given a particular substrate (P>0.05, Kruskal Wallis test). However, variability of BCAC01 in the HRP group suggests that greater variability, rather than release, is responsible for this result (FIG. 3)

TABLE 1
Key characteristics of ENGases, including those described in this work.

| Characteristic | PNGaseF | EndoBI-1 | AVUL01 | BCAC01 | BFIN01 |
|---|---|---|---|---|---|
| Organism of | Flavobacterium | Bifidobacterium | Alistipes | Bacteroides | Bacteroides |
| origin | meningosepticum | longum subsp. | onderdonkii | ovatus, | finegoldii |
| | | infantis | subsp. | B. | |
| | | | vulgaris | xylanisolvens | |
| Described by: | Tarentino and | Karav et al | This work | This work | This work |
| | Plummer 1994 | 2015 | | | |
| Modifications | ND¹ | ND | Anchor | Anchor | Anchor |
| made in this | | | domain | domain | domain |
| work | | | removed | removed | removed |
| ENGase | Reported by | ++ | +++ | +++ | +++ |
| Activity² | Tarentino and | | | | |
| | Plummer, 1994 | | | | |
| Optimum | 37° C. | 53° C. | 52° C. | 43° C. | 32° C. |
| Conditions: | pH 8.6 | pH 4.5 | pH 4.4 | pH 5.4 | pH 5.7 |
| Temperature | | | | | |
| pH | | | | | |
| Substrate | Active | Active | TBD | Active | Active |
| activity³: | Inhibited | Active | TBD | Active | <10%⁴ |
| bLF HRP | | | | | |

¹Not determined in this work;
²Activity is expressed as maximal, full conversion of RNAseB to its de-glycosylated form (+++) or relative decreasing levels of activity (++ or +);
³bLF is a complex N-glycan without α-1,3 core fucosylation, HRP is a high-mannose N-glycan with α-1,3 core fucosylation;
⁴Relative to maximum release of glycans under optimum conditions with bLF.

Discussion Glycoproteins are proteins with O- or N-conjugated glycans and are abundant in our diet. The glycan fraction of N-glycoproteins can represent as much as −8% of protein mass by weight, making N-glycans themselves an abundant component of our diet. However, because they are conjugated to proteins, N-glycans are poorly utilized by key gut bacteria and their nutritional value as fiber is hidden because they are not, in their native form, measured by AOAC methods.

Aside from cost, a major issue identified by the field is whether the inclusion of existing fiber additives like resistant starches (RS), galactooligosaccharides (GOS), polydextrose (PDX), or fructooligosaccharides (FOS) can provide broad benefits to the population. While these commercially available prebiotic fibers do generally appear to shape gut microbiome composition and metabolism, the effect is not universal across study participants. This is frequently referred to as ‘responders’ and ‘non-responders,’ where ‘responders’ have bacteria that can access the given fiber being studied. In contrast, the ‘non-responders’ fail to demonstrate a significant response (whether in terms of microbiome compositional or metabolic changes or changes to human health parameters). Some studies, though not all, do report tolerance issues stemming from the consumption of a large amounts of a single type of fiber, which could dissuade consumers from continuing fiber supplementation. This lack of a broad-spectrum effect and the difficulty in predicting these effects poses a major barrier to the widespread use of these purified or synthetic prebiotic fibers as a fiber supplement or fiber replacement.

It is important to note that our past work has already satisfied one of the two key qualifications needed to consider N-glycans as prebiotic fibers. To that end, we have shown that they survive digestion in the proximal human gut, that they can be released enzymatically, and we have shown that they can selectively enrich members of the human gut microbiome. Thus, N-glycans are a novel and promising source of complex, bioactive fiber. Currently, the production of these N-glycans is not trivial and there are limited opportunities for the enzymatic release of these N-glycans under different conditions (high vs. low temperature, high vs. low pH) and on distinct substrates (e.g., fucosylated vs. non-fucosylated N-glycan cores).

The novel enzymes described in this work (AVUL01, BCAC01, and BFIN01) represent three novel approaches to produce these enzymes under a variety of conditions for the production of food-grade N-glycans.

The present disclosure is not to be limited in terms to the particular examples described in this application, which are intended as illustrations of various aspects. Many modifications and variations can be made without departing from its spirit and scope, as will be apparent to those skilled in the art. Functionally equivalent methods within the scope of the disclosure, in addition to those enumerated herein, will be apparent to those skilled in the art from the foregoing descriptions. Such modifications and variations are intended to fall within the scope of the appended claims.

What is claimed is:

1. A recombinant protein comprising an amino acid sequence of SEQ ID NO: 4, SEQ ID NO: 5, or SEQ ID NO: 6.

2. The recombinant protein of claim 1, wherein the recombinant protein is an enzyme capable of releasing N-glycans from glycoproteins.

3. A method of using an enzyme comprising:
orally ingesting an enzyme and one or more foodstuffs to increase the released N-glycan content of the one or more foodstuffs during digestion, wherein the enzyme comprises an amino acid sequence of SEQ ID NO: 4, SEQ ID NO: 5, or SEQ ID NO: 6, and wherein the one or more foodstuffs comprise one or more glycoproteins.

4. A composition comprising the amino acid sequence of SEQ ID NO: 4, SEQ ID NO: 5, or SEQ ID NO: 6, and a glycoprotein.

5. The composition of claim 4, wherein the glycoprotein comprises high mannose.


SEQUENCE LISTING (9 sequences)

SEQ ID NO: 1 - DNA, 1377 bp, synthetic construct
  source 1..1377: mol_type = other DNA
gcctgcaccgacgacatcgagggcggcaagcctgtcgacgaaggggcatacgacgccgtgaagcgcgtcgacggcatgttgctcgacgtgacgtcgaacaagaacgagtcggtgatcgaactgcgctcggacaaataccagaccgaggtcttcttccgccttacgaaagcgccccagaagggtgtcgacgtgaagatcgaggtagatcccgcttatctggaaacttataatgccgagcacgggacggagttccagctcttccccgcggccgatgtcgccatcgaccgcgagggcaagctgctgctggccccggacgagatccgttcggtcggggtgggcgttaccctggccgcaggcagcgaattgcaggaggggacgacctatctgcttccgctcaaggcaacctcctcgaccgagggtatcacgctctcggaaaaggcgcagcatgcggtctatctggtgaaagacctgcgagcccagggcgacgcttacaagggccccgacgccgtgaggaacgtgtgctatttcgaggtcaacgacaccaacccgctcaatgcgctggagttcgtgctcaaggacagcggcaagctcttcttcgacgacatcatccttttttcggccaatatcaattacaatgcggaaacgggacgggtctatgtgctgaacaaccccaacgtgcagttcctgctggacaacaacgagcagttcctgcagccgttgcgcaagcgtggcatgaaggtgatcctcggtatcctgggcaaccacgatgccgccggcgtggcacagctgtccgatatgggctgccgcgagtttgccaaggagctggccgcttattgcaatgcctacaacctcgacggcgtcggtttcgacgacgagtattcgaactctcccgacctgagcaatccctggtttgcgccgtggtcgtcggctgccgcctcccggctgctctatgaaaccaagaaagccatgcccgataaaaccgtcatggtttactatctgggcggtatcaatagccggatgccttctgtcgacggggtcgagccgggcatgttcgtcgactatgccgtcgccgattacgggggaaccgcagcgccgatgcagggcatgaccgtcaagcagtgctccggcatgtcgatcgagttgaaccggggcagcggggattcgagcgagggcacagcccgctcgaagaaggaggccggatacggctattacatgttcttcgcactggggcataccaattcgttggtaagcgatttcagcgactatcggttccaggtgagccgttgccagtctgttgcccggggcctctatgacgaggaactgctgatgcccgagtattattacaaaaagaacgacgcaacgcgttataaactgcct

SEQ ID NO: 2 - DNA, 1350 bp, synthetic construct
  source 1..1350: mol_type = other DNA
gaggaagacattgaaataggaaacaagatagatgaatcttcttatttggcatcgactcaactgagcggattattattggacgaaaatacgaataaaaacagttcggtaatcgagctccgaaataatgaatatagcactaatgtggtattccgtctttctaaactacctcaaaaaggagtagatgttcaaatagctgtcgaggaatcatatgcagctatatacaacactatccatgaaacggatttcgaagtgtttcctgccgctaatgtgaaaatcgcaaataatggtacttttgtacttgccccagatgataaagttaccccaagcgtgaaagttacactgactgcgtttgatggaatggaagaggataaaacctacatagtacctttgacagtaacatcttctactgaaggagtaacatttactgaaacgtccaagcatatggtgcttctcgttcaagattatcgcaacaaacctaatactaacaaaggagaagatgcggtacaaacagttctttatttcgaagttaatgatactaacccgctcaatgcccttgagtttctgactgaaagtggtaaatattttttcgaccatatcgtactttttgctgctaatatcaattgggatccagagaaacaacgagtatatttagcgaacaatgaaaatgttcaattcttgctcgacaataatgataagtatctacaaccattgcgtaaagctggtatgaaaattattatcagcatccttggtaatcatgacgaagctggtgtcgctcaactttcggatatgggagctagggaattcgcccgggaactggcagcttattgccgtgcttataatctggacggcgtcgctttcgatgacgaatattcgaactcaccagacctttcaaacccctggcttgcaagcccatcagcttatgcaggttcacgattaatgtatgaatgtaaagctgtcatgccggaaaaaattgtctcgctctataaccttggcaacatgtattcaagtagtctccaagttatcgatggtatcgaacctggtcagtattgcgattatgctgtagctgactatggtggtgctgctgggccaggtaccggtatgacattgaaacagtgtgctggtatgtcaatagaactaagacgaggcagtggcaactcaagtgaatctacagcgcgttctagaaaagaagctggttatggatactacatgttcttcgcacttgacccatcattatatggttcacaagtatatagatgccaatccgtttgtaaaggtttgtacgatgaaacattagtttatccaagttactattataaaaagaatagtacagaacgtgaagctattaactga

SEQ ID NO: 3 - DNA, 1351 bp, synthetic construct
  source 1..1351: mol_type = other DNA
tgatgatttagagattggaaagcagtttgatgaatcggctttggagggaatttatgaaaatagagtttttctttgcgacgcttcatctaataaagtcaataatattattgaattatatacttctaatcagtattcaactaaagttcgaatgtctttatcgaagaattcttcagctgaaacttcagctaagatagttatagatgaatcttatttggaaaactataatcaagaacataacacagattttgaaatgcttccggcaagtgcggtcatttttgctaatgatggtactttaaactctggaaattgtggaagaagtttagaggtggaaatgacaataaagatttcagatgatttggaggcaggaaagacatatgctttacctctctcaataacggaacctacttctgaccttacaataaaaaatgagcaagatcgtcactgtgtatatttagttaaggacatgagaaagtttggtgacgctttcaaaggagaggataagccaaaaggatttttgttttttgaagtaaatgatgtaaatccattaaatgctctttctttccaattggagaacggtaaatatttttgggatgtagttgttttatttgctgctaatatcaactatgattctgaagcagggcgtccatatataaaatgtaatccgaatgttcagtatttgctagataataatgagacacttttgcagccattgcgtaaacgcggtataaaagttttgttaggtttattgggtaatcatgatatggcaggattggctcaactatctactgagggggctaaagtatttgcgcgtgaggttgctcaatattgcaaggcttataacttggatggagtaaattatgatgatgaatattcgctttatccagatcttggtaatccttcatttgctactccttctgctcaagcgggagcacgtctctgctatgaaaccaaacaggtaatgcctgataagttagttacagtttttgcgtatgggaacatgtacggagttgaatctgtagatggagtggatgttgatgagtggattgatatagttgtacctaattatggttcagcagcttatcctttaggtaatatggataaatggaagtgtgcaggtttggctgcagaatttaatttgcatggtggtggctctttggatagttatggcgctcagaaaatattgagtggtggttatggatggcatatgggatttgctgctaatccgaataattatacaactatttttcaacgtttgagcggtgcttatgaattatatggctctaaattgaaagatccgacaatcttttataagaaaaatgaaactactccatctaagtatccggaggattta

SEQ ID NO: 4 - protein, 473 aa, synthetic construct
MHHHHHHENLYFQGACTDDIEGGKPVDEGAYDAVKRVDGMLLDVTSNKNESVIELRSDKYQTEVFFRLTKAPQKGVDVKIEVDPAYLETYNAEHGTEFQLFPAADVAIDREGKLLLAPDEIRSVGVGVTLAAGSELQEGTTYLLPLKATSSTEGITLSEKAQHAVYLVKDLRAQGDAYKGPDAVRNVCYFEVNDTNPLNALEFVLKDSGKLFFDDIILFSANINYNAETGRVYVLNNPNVQFLLDNNEQFLQPLRKRGMKVILGILGNHDAAGVAQLSDMGCREFAKELAAYCNAYNLDGVGFDDEYSNSPDLSNPWFAPWSSAAASRLLYETKKAMPDKTVMVYYLGGINSRMPSVDGVEPGMFVDYAVADYGGTAAPMQGMTVKQCSGMSIELNRGSGDSSEGTARSKKEAGYGYYMFFALGHTNSLVSDFSDYRFQVSRCQSVARGLYDEELLMPEYYYKKNDATRYKLP

SEQ ID NO: 5 - protein, 463 aa, synthetic construct
MHHHHHHENLYFQGEEDIEIGNKIDESSYLASTQLSGLLLDENTNKNSSVIELRNNEYSTNVVFRLSKLPQKGVDVQIAVEESYAAIYNTIHETDFEVFPAANVKIANNGTFVLAPDDKVTPSVKVTLTAFDGMEEDKTYIVPLTVTSSTEGVTFTETSKHMVLLVQDYRNKPNTNKGEDAVQTVLYFEVNDTNPLNALEFLTESGKYFFDHIVLFAANINWDPEKQRVYLANNENVQFLLDNNDKYLQPLRKAGMKIIISILGNHDEAGVAQLSDMGAREFARELAAYCRAYNLDGVAFDDEYSNSPDLSNPWLASPSAYAGSRLMYECKAVMPEKIVSLYNLGNMYSSSLQVIDGIEPGQYCDYAVADYGGAAGPGTGMTLKQCAGMSIELRRGSGNSSESTARSRKEAGYGYYMFFALDPSLYGSQVYRCQSVCKGLYDETLVYPSYYYKKNSTEREAIN

SEQ ID NO: 6 - protein, 1351 aa, synthetic construct
TGATGATTTAGAGATTGGAAAGCAGTTTGATGAATCGGCTTTGGAGGGAATTTATGAAAATAGAGTTTTTCTTTGCGACGCTTCATCTAATAAAGTCAATAATATTATTGAATTATATACTTCTAATCAGTATTCAACTAAAGTTCGAATGTCTTTATCGAAGAATTCTTCAGCTGAAACTTCAGCTAAGATAGTTATAGATGAATCTTATTTGGAAAACTATAATCAAGAACATAACACAGATTTTGAAATGCTTCCGGCAAGTGCGGTCATTTTTGCTAATGATGGTACTTTAAACTCTGGAAATTGTGGAAGAAGTTTAGAGGTGGAAATGACAATAAAGATTTCAGATGATTTGGAGGCAGGAAAGACATATGCTTTACCTCTCTCAATAACGGAACCTACTTCTGACCTTACAATAAAAAATGAGCAAGATCGTCACTGTGTATATTTAGTTAAGGACATGAGAAAGTTTGGTGACGCTTTCAAAGGAGAGGATAAGCCAAAAGGATTTTTGTTTTTTGAAGTAAATGATGTAAATCCATTAAATGCTCTTTCTTTCCAATTGGAGAACGGTAAATATTTTTGGGATGTAGTTGTTTTATTTGCTGCTAATATCAACTATGATTCTGAAGCAGGGCGTCCATATATAAAATGTAATCCGAATGTTCAGTATTTGCTAGATAATAATGAGACACTTTTGCAGCCATTGCGTAAACGCGGTATAAAAGTTTTGTTAGGTTTATTGGGTAATCATGATATGGCAGGATTGGCTCAACTATCTACTGAGGGGGCTAAAGTATTTGCGCGTGAGGTTGCTCAATATTGCAAGGCTTATAACTTGGATGGAGTAAATTATGATGATGAATATTCGCTTTATCCAGATCTTGGTAATCCTTCATTTGCTACTCCTTCTGCTCAAGCGGGAGCACGTCTCTGCTATGAAACCAAACAGGTAATGCCTGATAAGTTAGTTACAGTTTTTGCGTATGGGAACATGTACGGAGTTGAATCTGTAGATGGAGTGGATGTTGATGAGTGGATTGATATAGTTGTACCTAATTATGGTTCAGCAGCTTATCCTTTAGGTAATATGGATAAATGGAAGTGTGCAGGTTTGGCTGCAGAATTTAATTTGCATGGTGGTGGCTCTTTGGATAGTTATGGCGCTCAGAAAATATTGAGTGGTGGTTATGGATGGCATATGGGATTTGCTGCTAATCCGAATAATTATACAACTATTTTTCAACGTTTGAGCGGTGCTTATGAATTATATGGCTCTAAATTGAAAGATCCGACAATCTTTTATAAGAAAAATGAAACTACTCCATCTAAGTATCCGGAGGATTTA

SEQ ID NO: 7 - DNA, 243 bp, synthetic construct
  source 1..243: mol_type = other DNA
atgggcagcagccatcatcatcatcatcacagcagcggcctggtgccgcgcggcagcaagatcgagtgcaagatcatcaacaccgaggtctacaccatcgaggacatcaagaagaagaacggcatcgaggccctcaagatcatctgcaagatctgccgcggcctggaggtcatcgacgacgaggtcatctgcgagatcaagaaggaggtcgaggaggtcaacgagatcatcgagaagctgtga

SEQ ID NO: 8 - DNA, 720 bp, synthetic construct
  source 1..720: mol_type = other DNA
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccccggaattcccgggtcgactcgagcggccgcatcgtgactga

SEQ ID NO: 9 - DNA, 42 bp, synthetic construct
  source 1..42: mol_type = other DNA
atgcatcatcatcatcatcatgaaaacctgtattttcagggc